(19) United States
(12) Patent Application Publication
(10) Pub. No.: US 2012/0028825 A1
(43) Pub. Date: Feb. 2, 2012

(54) GALECTIN-1 (GAL1) AS A BIOMARKER FOR DIFFERENTIAL DIAGNOSIS OF OSTEOSARCOMA AND CHONDROSARCOMA

(76) Inventors: Anne Gomez-Brouchet, Toulouse (FR); Claudine Schiff, Marsellie Cedex (FR)

(21) Appl. No.: 13/148,113

(22) PCT Filed: Feb. 10, 2010
(86) PCT No.: PCT/EP10/51640

§ 371 Date: Aug. 5, 2011

(51) Int. Cl. C40B 30/04; C12Q 1/68; G01N 33/566

(52) U.S. Cl. 506/9; 435/7.23; 435/6.11; 435/6.12

(57) ABSTRACT

The present invention relates to a method for differential diagnosis of osteosarcoma and chondrosarcoma, especially chondroblastic osteosarcoma and conventional chondrosarcoma, in a patient comprising a step consisting of detecting galectin-1 (GAL1) expression in a bone sample obtained from said patient.

### FIELD OF THE INVENTION

The invention relates to galectin-1 (GAL1) as a biomarker for differential diagnosis of osteosarcoma and chondrosarcoma.

### BACKGROUND OF THE INVENTION

The most common type of primary malignant bone tumor is osteosarcoma, which produces immature bone. Chondrosarcoma represents another type of bone cancer, which generates cartilage. The diagnosis of osteosarcoma is based on the identification of osteoid by osteoblastic tumor cells. High-grade osteosarcoma (including conventional osteosarcoma, telengiectatic osteosarcoma and small cell osteosarcoma) and low-grade osteosarcoma (including paraosteal osteosarcoma and intramedullary well differentiated osteosarcoma) are diagnosed on histological features. Among conventional osteosarcomas, osteoblastic, chondroblastic and fibroblastic sub-groups are defined, according to the abundance and the nature of the matrix produced by the tumor cells. Chondroblastic osteosarcoma, for example, is characterized by the presence of a predominantly chondroid matrix, i.e. a matrix that resembles cartilage, in addition to pockets of osteoid matrix (Fletcher C D M et Al., 2002). Chondrosarcomas, on the other hand, are diagnosed by the presence of chondroid matrix in foci or throughout the tumor. They are subdivided into conventional, mesenchymal, clear cell and dedifferentiated chondrosarcomas. Conventional chondrosarcomas are the most prevalent.

Because chondroblastic osteosarcoma produces chondroid as well as osteoid matrix, it is sometimes difficult to diagnose by current microscopic methods. In small biopsy samples, the osteoid component of chondroblastic osteosarcoma may not be present, leading to an ambiguous diagnosis of either chondroblastic osteosarcoma or conventional chondrosarcoma. Distinguishing an osteosarcoma from a chondrosarcoma is very important to perform since this can have important consequences for the therapeutic management of these patients as the treatments and prognoses differ significantly.

Conventional osteosarcoma is largely a disease of the young (second decade of life) but 30% of osteosarcomas also occur in patients over 40 years of age. Without any treatment, prognosis is poor, due to the development of pulmonary metastasis. It has been improved by the combination of neo-adjuvant chemotherapy and a conservative surgery of the affected member (Fletcher C D M et Al., 2002; Huvos, A G et Al., 1977; Rosen, G et Al., 1982; Picci, P. 2007). On the other hand, all forms of chondrosarcomas affect mainly patients over 50 years, but are not rare in younger individuals (Koh, J S et Al., 2001; Gupta, K et Al., 2004; Mahesha, V et Al., 2006). In contrast to osteosarcomas, the prognosis is less pejorative and depends on the histological grade of the tumor. The surgery is the only efficient treatment. However, radiotherapy and/or chemotherapy are sometimes added in case of aggressive form of chondrosarcomas as in the dedifferentiated or the mesenchymatous forms (Fletcher C D M et Al., 2002). The diagnosis of osteosarcomas or chondrosarcomas, essentially based on morphological parameters, depends on the quality of the surgical biopsy. There is no diagnostic marker to discriminate accurately between the 2 types of tumors. Transcription factors (Runx2, osterix, (β-catenin, Msx1, 2, Dlx5-6, Twist, AP1, Knox-20, Sp3 and ATF4) involved in osteoblast differentiation (Komori, T. 2006), and Sox family transcription factors (Sox 9, 5, 6) involved in the regulation of chondrocyte differentiation (Mackie, E J et Al., 2008), are not lineage specific. None of these markers could be used as diagnostic tools.

So, there is a permanent need in the art for a method for differential diagnosis of osteosarcoma and chondrosarcoma.

Galectins are a large family of calcium independent S-type lectins, widely conserved in animals, plants and microorganisms, which bind to β-galactose derivatives (Drickamer, K. 1988; Sharon, N., et Al., 1989; Bourne, Y., et Al., 1994). Functional studies have implicated galectins in cell growth and cell cycle progression (Sanford G L, et Al., 1990), in cell differentiation and apoptosis (Ellerhorst J, et Al., 1999; Rorive S, et Al., 2001; Koh H S, et Al., 2008; Brandt B, et Al., 2008; Valenzuela H F et Al., 2007), in cell adhesion (Moiseeva E P, et Al., 1999 and Van den Brûle F et Al., 2004), and in cell migration (Camby I, et Al., 2002 and Elola M T, et Al., 2007). Galectins can also suppress cell growth depending on the cells, the receptors types and the doses used (Kopitz J, et Al., 2001 and Andersen H, et Al., 2003). To date, fifteen galectins have been identified in mammals (Barondes S H et Al., 1994), among which galectin-1 (GAL1) was found abundantly expressed by many cell types, such as skeletal, smooth and cardiac muscle and from other cells of mesenchymal origin (Camby I, et Al., 2006). In human tumor cells, GAL1 is overexpressed by endothelial cells and played a crucial role in tumor angiogenesis (Thijssen V L, et Al., 2006). It was also expressed by human breast cancer, colon cancer and glioma cell lines (Satelli A, et Al., 2008). Moreover, human urothelial (Cindolo, L, et Al., 1999) and thyroid carcinoma (Chiariotti, L, et Al., 1995 and Xu, X C, et Al., 1995), endometrial adenocarcinoma and breast carcinoma overexpressed GAL1 compared to the corresponding normal cells (Jung E J, et Al., 2007).

However, GAL1 has never been proposed as a biomaker for differential diagnosis of osteosarcoma and chondrosarcoma.

### SUMMARY OF THE INVENTION

The invention relates to a method for differential diagnosis of osteosarcoma and chondrosarcoma, especially between chondroblastic osteosarcoma and conventional chondrosarcoma, in a patient comprising a step consisting of detecting galectin-1 (GAL1) expression in a bone sample obtained from said patient.

### DETAILED DESCRIPTION OF THE INVENTION

The invention relates to a method for differential diagnosis of osteosarcoma and chondrosarcoma, especially between chondroblastic osteosarcoma and conventional chondrosarcoma, in a patient comprising a step consisting of detecting galectin-1 (GAL1) expression in a bone sample obtained from said patient.

As used herein, the term “Galectin-1” or “GAL1” has its general meaning in the art and denotes a member of the galectins family which was found abundantly expressed by many cell types, such as skeletal, smooth and cardiac muscle and from other cells of mesenchymal origin. An exemplary sequence for human GAL1 protein is deposited in the database under accession number NM 002305.3.

Typically, the bone sample according to the patient is a bone biopsy, more particularly a biopsy of a bone tumor, even more particularly a biopsy of a bone tumor comprising tumor cells.

In a particular embodiment, the method of the invention allows differential diagnosis of chondroblastic osteosarcoma and conventional chondrosarcoma.

The term “detecting” as used above includes qualitative and/or quantitative detection (measuring levels) with or without reference to a control. Typically GAL1 expression may be measured for example by RT-PCR or immunohistochemistry performed on said bone sample.

For example detecting GAL1 expression in said bone sample may be performed by determining the expression level of GAL1 gene.

Typically, the determination comprises contacting the sample with selective reagents such as probes, primers or ligands, and thereby detecting the presence, or measuring the amount, of polypeptides or nucleic acids of interest originally in the sample. Contacting may be performed in any suitable device, such as a plate, microtiter dish, test tube, well, glass, column . . . . In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a particular embodiment, the expression level may be determined by determining the quantity of mRNA. Such method may be suitable to determine the expression level of GAL1 gene in the bone sample.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA may be then detected by hybridization (e.g., Northern blot analysis).

Alternatively, the extracted mRNA may be subjected to coupled reverse transcription and amplification, such as reverse transcription and amplification by polymerase chain reaction (RT-PCR), using specific oligonucleotide primers that enable amplification of a region in the GAL1 gene. Preferably quantitative or semi-quantitative RT-PCR is preferred. Real-time quantitative or semi-quantitative RT-PCR is particularly advantageous. Extracted mRNA may be reverse-transcribed and amplified, after which amplified sequences may be detected by hybridization with a suitable probe or by direct sequencing, or any other appropriate method known in the art.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

In a particular embodiment, the method of the invention comprise the steps of providing total RNAs obtained from the bone sample of the patient, and subjecting the RNAs to amplification and hybridization to specific probes, more particularly by means of a quantitative or semi-quantitative RT-PCR.

Total RNAs can be easily extracted from the bone sample. For instance, the bone sample may be treated prior to its use, e.g. in order to render nucleic acids available. Techniques of cell or protein lysis, concentration or dilution of nucleic acids, are known by the skilled person.

In another embodiment, the expression level may be determined by DNA microarray analysis. Such DNA microarray or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Detection of GAL1 in the bone sample may also be performed by determining the expression level of GAL1 protein.

Such methods comprise contacting a bone sample with a binding partner capable of selectively interacting with GAL1 present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A bone sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

One preferred method utilizes immunohistochemistry, a staining method based on immunoenzymatic reactions using monoclonal or polyclonal antibodies to detect cells or specific proteins such as tissue antigens. Typically, immunohistochemistry protocols involve at least some of the following steps:
1) antigen retrieval (e.g., by pressure cooking, protease treatment, microwaving, heating in appropriate buffers, etc.);
2) application of primary antibody (i.e. anti-GAL1 antibody) and washing;
3) application of a labeled secondary antibody that binds to primary antibody (often a second antibody conjugate that enables the detection in step 5) and wash;
4) an amplification step may be included;
5) application of a detection reagent (e.g. chromagen, fluorescently tagged molecule or any molecule having an appropriate dynamic range to achieve the level of or sensitivity required for the assay);
6) counterstaining may be used and
7) detection using a detection system that makes the presence of the proteins visible (to either the human eye or an automated analysis system), for qualitative or quantitative analyses.

Various immunoenzymatic staining methods are known in the art for detecting a protein of interest. For example, immunoenzymatic interactions can be visualized using different enzymes such as peroxidase, alkaline phosphatase, or different chromogens such as DAB, AEC, or Fast Red; or fluorescent labels such as FITC, Cy3, Cy5, Cy7, Alexafluors, etc. Counterstains may include H&E, DAPI, Hoechst, so long as such stains are compatable with other detection reagents and the visualization strategy used. As known in the art, amplification reagents may be used to intensify staining signal. For example, tyramide reagents may be used. The staining methods of the present invention may be accomplished using any suitable method or system as would be apparent to one of skill in the art, including automated, semi-automated or manual systems.

The method of the invention may comprise a further step consisting of comparing GAL1 expression with a control reference.

In a particular embodiment, when detecting of GAL1 expression is performed by immunochemistry, the method may further comprise a step consisting of determining the amount of cells that express GAL1 (“GAL1+ cells”).

In a preferred embodiment a pourcentage of GAL+ cells of at least 20%, preferably at least 21%, more preferably at least 22%, even more preferably at least 23%, at least 24%, at least 25%, at least 26%, at least 27%, preferably at least 28%, more preferably at least 29%, even more preferably 30% and more preferably 50% is indicative of osteosarcoma.

In a particular embodiment, the step of determining the amount of GAL1+ cells may be combined with a step of determining the staining intensity of the GAL1+ cells.

The present invention also relates to kits for differential diagnosis of osteosarcoma and chondrosarcoma comprising means for detecting GAL1 expression.

According to the invention, the kits of the invention may comprise an anti-GAL1 antibody; and another molecule coupled with a signalling system which binds to said GAL1 antibody.

Typically, the antibodies or combination of antibodies are in the form of solutions ready for use. In one embodiment, the kit comprises containers with the solutions ready for use. Any other forms are encompassed by the present invention and the man skilled in the art can routinely adapt the form to the use in immunohistochemistry.

The present invention also relates to galectin-1 (GAL1) as a biomarker for differential diagnosis of osteosarcoma and chondrosarcoma.

The present invention also relates to galectin-1 (GAL1) as a biomarker for osteoblasts, especially human osteoblasts.

In another embodiment, the invention relates to a method for distinguish between osteosarcoma and chondrosarcoma in a patient comprising a step consisting of detecting galectin-1 (GAL1) expression in a bone sample obtained from said patient.

In a particular embodiment, the method allows a distinguishement between chondroblastic osteosarcoma and conventional chondrosarcoma.

The present invention also relates to galectin-1 (GAL1) as a biomarker for distinguishing osteosarcoma from chondrosarcoma.

The invention will further be illustrated in view of the following figures and examples.

### FIGURES

FIG. 1: Immunohistochemical staining for GAL1 in benign bone proliferations.

Sections were prepared from two different types of benign proliferation, periostitis (A-D) and osteoblastoma (E-H). The sections were either stained with H&E (A, B, E and F) or immunostained for GAL1 (C, D, G and H), and photographed at ×200 magnification (A, C, E and G) or at ×400 magnification (B, D, F and H). In periostitis, mature woven trabeculae are lined with proliferating fibroblastic osteoblasts and are present in a vascular and loosely fibrous stroma (A, B); the proliferating osteoblasts present an intense GAL1 staining (C, D). In osteoblastoma, the tumor is highly cellular and osteoid trabeculae show prominent osteoblastic rimming; osteoblasts are plump with uniform nuclei (E, F). Intense GAL1 staining is seen in plump osteoblasts (G, H). Arrows point to the osteoblasts of the osteoblastic rimming in periostitis (stained or not for GAL1) and the plump osteoblasts of the osteoblastoma (stained or not for GAL1).

FIG. 2: Morphological characteristics of malignant bone tumors and their immunohistochemical staining for GAL1.

Sections of TMAs (A-K) containing several types of malignant bone tumor: grade II conventional chondrosarcoma (A, B, C); grade III conventional chondrosarcoma (D, E, F); high-grade chondroblastic osteosarcoma (G, H, I), and dedifferentiated chondrosarcoma (J, K) as well as a papillary thyroid adenocarcinoma (L). Sections were stained with H&E and photographed at ×200 magnification (A, D, G, J) and at ×400 magnification (B, E, H). Sections stained for GAL1 were photographed at ×200 magnification (C, F, I, K, L). The matrix is chondroid in A, B, G and H and chondro-myxoid in D and E. Grade II chondrosarcoma (A, B) is characterized by moderate cell content and chondrocytes with a moderate degree of nuclear atypia. Grade III chondrosarcoma (D, E) is highly cellular and chondrocytes show greater atypia than grade II chondrosarcoma. In this sample of chondroblastic osteosarcoma (G, H), no osteoid matrix is visible. Cells are morphologically similar to chondrocytes and present moderate to high atypia. Ninety percent of osteoblasts present intense GAL1 staining (I) whereas chondrocytes are negative for GAL1 staining whatever the grade of the chondrosarcoma (C, F). Dedifferentiated chondrosarcoma (J) contains two clearly defined components: a well differentiated chondrosarcoma (left) and a high-grade osteosarcoma (right). The arrow points to osteoid produced by osteosarcoma cells. The low-grade chondrosarcoma component • is not stained for GAL1 (left) whereas the osteosarcoma component ♦ (right) is highly stained (K). The arrow points to staining for GAL1 in osteosarcoma cells. Papillary thyroid carcinoma serves as a positive control for GAL1 staining (L). Arrows point to adenocarcinoma cells that stain strongly for GAL1.

FIG. 3: Western blot analysis of GAL1 expression in chondrosarcoma and osteosarcoma.

Western blotting was performed using rabbit anti-GAL1 and anti-GAPDH antiserum, revealed with HRP-coupled protein-A on 2 representative cases of osteosarcoma (OS) and chondrosarcoma (CS). For GAL1 detection, human osteoblasts cells (OB) and human mesenchymal stem cells (MSC) serve as positive controls and GAL1 deficient stromal cells as a negative control (Ctl).

### EXAMPLE

### Material & Methods

Patient and tumor characteristics: Patient's characteristics and tumor location are presented in Table 1.

TABLE 1
Characteristics of patients and tumors.

| | | | | | Statistical |
|---|---|---|---|---|---|
| | | OS | CS | All tumors | values |
| | Number | 87 | 78 | 165 | |
| Male | Number | 45 | 49 | 94 | P = 0.15 |
| | % | 52 | 63 | 57 | |
| Median age | Years | 16 | 58 | 21 | P < 10⁻⁴ |
| | IQR | 13-23 | 44-73 | 15-54 | |
| Flat bone | Number | 11 | 38 | 44 | |
| | % | 13 | 49 | 27 | P < 10⁻⁴ |
| Long bone | Number | 76 | 40 | 116 | |
| | % | 87 | 51 | 73 | |

OS and CS stand for osteosarcoma and chondrosarcoma, respectively. Given the non-Gaussian distribution of age, Inter Quartile Range (IQR) values are reported. IQR=p50-(p25-p75) where p50 is the median and p25 and p75 the first and third quartiles, respectively. The age between OS and CS is compared using the non-parametric test of Wilcoxon. Gender distribution and tumor localization in bone between OS and CS are compared using chi-square tests. P-values are given uncorrected.

One hundred and sixty-five osseous sarcomas, from 3 French departments of pathology (Rangueil Hospital, Toulouse; La Timone Hospital, Marseille; Cochin Hospital, Paris) were studied. Eighty-seven cases are osteosarcomas and 78 are chondrosarcomas. All osteosarcomas are of high-grade, 86 are conventional osteosarcomas and one is a telengiectatic osteosarcoma. Among the conventional osteosarcomas, 39 correspond to osteoblastic osteosarcomas, 25 to chondroblastic osteosarcomas, 14 to fibroblastic osteosarcomas and 9 cannot be assigned to a specific sub-groups due to special morphological aspects (with predominant giant cells or with heavily calcified matrix). Among chondrosarcomas, 66 are conventional chondrosarcomas (19, 41 and 3 of grade I, II and III, respectively), 7 are mesenchymatous chondrosarcomas (grade III), 2 are clear cell chondrosarcomas (grade I) and 3 are dedifferentiated chondrosarcomas (grade III). Histological diagnosis has been made (on the initial biopsy and secondarily on the surgical resection), by 9 expert pathologists of the French Group of Bone Pathologists (GFPO) before this study. Two benign reactive osseous proliferations (periostitis) and 5 osteoblastomas (benign bone tumor) have also been analyzed.

Construction of Tissue Microarrays (TMA):

Tissue microarrays (TMAs) were made by the three French pathology centers (at Toulouse, Marseille and Paris, as above) from 165 selected surgical biopsies, according to the TMA technique developed by Kononen et al. (Kononen J et al, 1998). Briefly, tissues samples were fixed in 10% buffered formalin, decalcified and embedded in paraffin wax. Representative areas of each tumor were selected for TMA production by first observing the biopsy tumor slide stained with hematoxylin and eosin (H&E) and then collecting the tissue from the corresponding paraffin blocks. A tissue microarray instrument (Beecher Instruments, Sun Prairie, Wis., USA) was used to create holes in the different paraffin blocks and, for each tumor, three tissue cores of 1-2 mm diameter were taken from the primary paraffin block and placed in a new paraffin block. Serial sections of 4 μm thickness were cut from the TMA blocks by using a conventional microtome. One section was stained immediately with H&E, whereas the remaining sections were stored at room temperature before immunostaining.

We did not need the approval of the French equivalent of the Institutional Review Board (Comité Consultatif de Protection des Personnes dans la Recherche Biomédiacle; CCPPRB) for this investigation since archived paraffin blocks are used routinely for diagnosis.

Immunohistochemistry

To minimize tissue loss during immunohistochemical processing, sets of 4-μm-thick sections from the TMA blocks were transferred to adhesive-coated slides. Standard immunoperoxidase procedures were used for immunohistochemistry. Briefly, the 4 μm TMA sections containing samples of the 165 bone tumors were deparaffinized and rehydrated. Sections were incubated with EDTA buffer (1 mM, pH 8) for 40 min at 98° C. to renature the antigen, then for 5 min in 3% hydrogen peroxide to quench endogenous peroxidase activity. Next, sections were incubated with mouse anti-human GAL1 mAb (Clone 25C1; Vector Laboratories, Burlingame, Calif., USA) at 1:200 dilution for 60 min at room temperature, followed by incubation first with biotinylated link antibody and then with peroxidase-labeled streptavidin (LSAB™+ Kit; Dako, Trappes, France). Staining was completed by incubation with diaminobenzidine solution (DAB liquid; Dako). For a negative control, the anti-GAL1 mAb was replaced with PBS; thyroid adenocarcinoma sections were used as positive controls for GAL1 staining.

The percentage of GAL1-positive cells and the staining intensity were estimated semi-quantitatively: six groups were made according to the percentage of stained cells (0; 1-5; 6-25; 26-50; 51-75; 76-100%) and four according to the staining intensity (no detectable (0), faint (+), moderate (++) and intense (+++) staining). Two osseous pathologists independently evaluated the percentage of stained cells and the staining intensity.

For immunohistochemistry of samples of periostitis and osteoblastoma, sections were cut from paraffinblocks and processed as for the TMA blocks.

Western Blotting

Two biopsies of chondroblastic osteosarcomas and conventional chondrosarcomas were sonicated in 1% IGEPAL CA-630, Tris 20 mM, PH 7.5, NaCl 150 mM, EDTA 1 mM and protease inhibitor mixture [Sigma Aldrich, L'Isle d'Abeau, France]). Human mesenchymal stem cells (a kind gift of K. Tarte and P. Ame-Thomas), human osteoblasts (PromoCell, Heidelberg, Germany) and GAL-1-deficient mouse stromal cells from our laboratory (30×10⁶ cells/ml per samples) were lysed directly in the IGEPAL lysis buffer. Proteins were quantified by using the colorimetric Bradford Protein Assay (Bio-Rad, Munich, Germany). Lysates containing 50 μg of protein were separated by SDS-PAGE on 15% acrylamide gels followed by Coomassie blue staining. Proteins were transferred onto 0.2 μm Immobilon-P membranes (Millipore, Bedford, Mass., USA) in 20% methanol, 25 mM Tris and 0.2 M glycine. Membranes were saturated with 5% wt/vol nonfat powdered milk in PBS containing 0.05% vol/vol Tween 20. GAL1 was detected by using a rabbit anti-GAL1 antiserum (reacting also with mouse GAL1) prepared in our laboratory ( 1/1000 dilution), and revealed by horseradish peroxidase-coupled protein A, followed by chemiluminescence detection (ECL, Amersham, Les Ulis, France), as previously described (Camby I. et al 2006). Membranes were stripped and then incubated with anti-actin mAb (Abcam, Paris, France) at 1/10 000 dilution and revealed by horseradish peroxidase-conjugated goat anti-mouse IgG (Sigma Aldrich).

All samples were used in compliance with the French bioethics laws regarding patient information and consent.

Statistical Analysis

The significance of the association between staining (percentage of stained cells and intensity of staining), and the tumor type was assessed by applying a Pearson's homogeneity chi-square test. The Cochran-Armitage Trend test was used to evaluate the significance of the association between osteosarcomas and the increase of the staining categories. The differential diagnostic value of GAL1 staining in distinguishing chondroblastic osteosarcomas from conventional chondrosarcomas was defined by its sensitivity and its specificity, for each staining category. Confidence intervals were given at 95%. Similar statistics were reported to distinguish chondroblastic osteosarcomas from conventional chondrosarcomas. Sensitivity (Se) is the probability that an osteosarcoma (or a chondroblastic osteosarcoma) is positive for GAL1 according to the staining criteria. Specificity (Sp) is the probability that a chondrosarcoma (or a conventional chondrosarcoma) is negative for GAL1 according to the staining criteria. The receiver operator characteristic (ROC) curve is a graphic representation of the relationship between the Se and the Sp of a test, calculated for all possible cut-off values. It provides a measure of the diagnostic performance of a test and allows comparison of the performance of several tests. The area under the ROC (AUROC) is an overall estimator of the test capacity to distinguish an osteosarcoma (or a chondroblastic osteosarcoma) from a chondrosarcoma (or a conventional chondrosarcoma). AUROC lies between 0 and 1 where 1, 0.5 and 0 correspond to a perfect, a non-informative and a confusing test, respectively.

The relevance of the proposed diagnostic test (see the Results section) was checked by determination of the positive (PPV) and negative (NPV) prediction values. PPV is the probability that the sample is an osteosarcoma (or a chondroblastic osteosarcoma) if the diagnostic test is positive. NVP is the probability that the sample is a chondrosarcoma (or a conventional chondrosarcoma) if the diagnostic test is negative. P-values are given uncorrected and a significance threshold of 0.05 was applied. Statistical analyses were performed using Stat 9.2 SE on the TIERS-Mip computational platform.

Results

Morphological Aspects and GAL1 Expression in Benign Osseous Proliferations

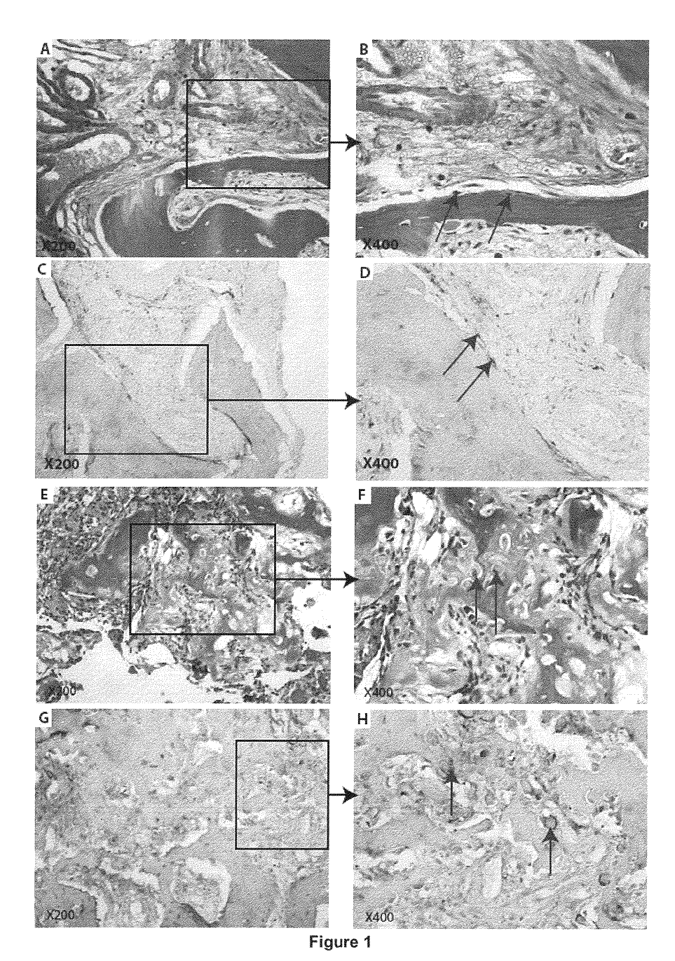

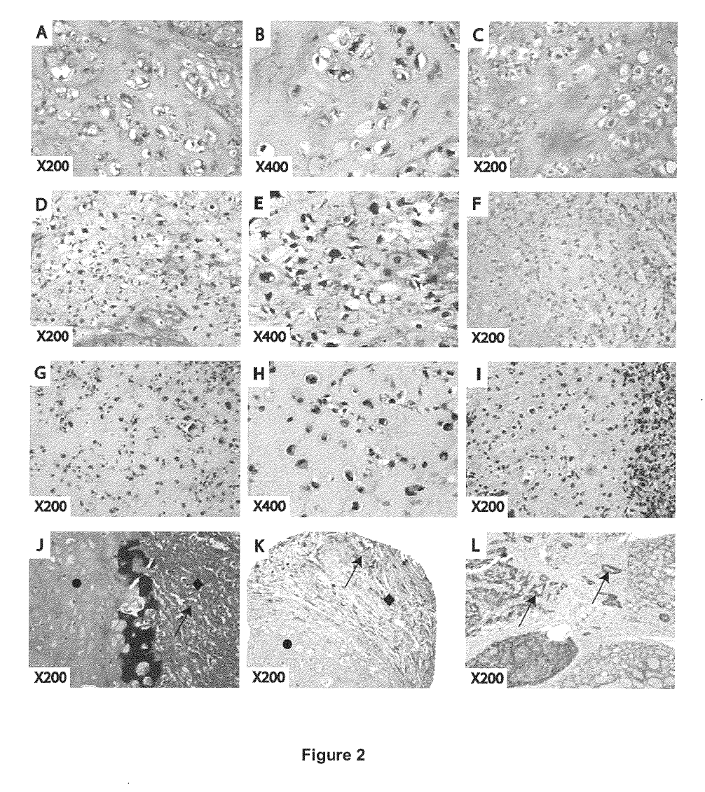

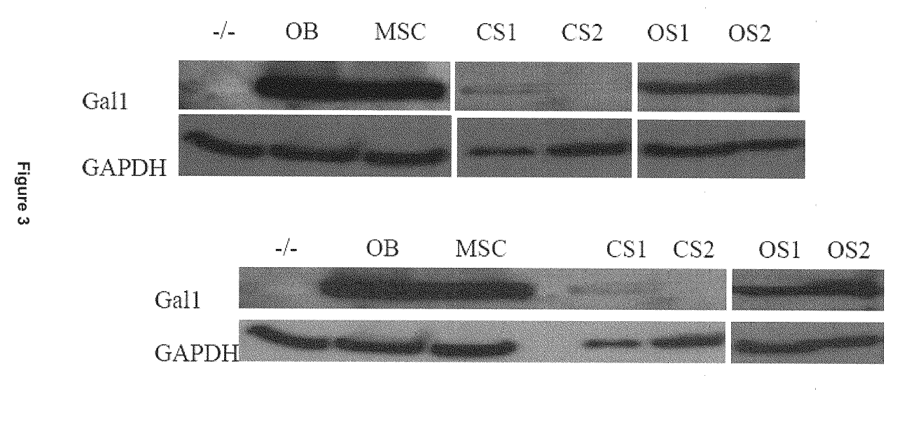

As no data were available concerning GAL1 expression in normal human osteoblasts, we analyzed two types of benign bone proliferation—periostitis and osteoblastomas (FIG. 1)—in which there is intense benign osteoblastic activation. A periostitis is an osteoblastic proliferation with fibrosis, mostly secondary to inflammation. As determined by H&E staining, the tissue reaction produces a dominant osseous component of mature woven bone with intense osteoblastic rimming (FIGS. 1A, B). Osteoblastomas are benign osteoblastic neoplasms characterized by a fibrovascular tissue with interlacing osteoid trabeculae, which are lined by plump osteoblasts (FIGS. 1E, F). GAL1 immunostaining was seen in the osteoblasts in both cases of periostitis (FIGS. 1C, D) and in all five cases of osteoblastoma (FIGS. 1 G, H), indicating that GAL1 is expressed in human osteoblasts. The staining was cytoplasmic and/or sometimes nuclear and was most intense close to the cell membrane of the majority of osteoblasts.

Morphological Aspects and GAL1 Expression in Chondroblastic Osteosarcomas and Conventional Chondrosarcomas

The clinical characteristics (age, gender and tumor localization) of the 165 sarcoma patients who participated in this study (gender, age and tumor localization) are presented in Table 1. The characteristics are consistent with other published data for these diseases. When the cases of osteosarcoma (87 samples) were compared to cases of chondrosarcoma (78 samples), no correlation with gender was found (p=0.15); osteosarcomas occurred more often than chondrosarcomas among young patients (p<10⁻⁴), and osteosarcomas were localized in long bones more frequently than were chondrosarcomas (p<10⁻⁴).

We also collected the clinical, radiological and anatomopathologic characteristics of all 165 tumors (data not shown). Among the 87 osteosarcomas, 25 were chondroblastic osteosarcomas and among the 78 chondrosarcomas, 66 were conventional chondrosarcomas (see Materials and Methods). FIG. 2 shows representative morphological views of a grade II and a grade III conventional chondrosarcoma (FIGS. 2A-C and D-F, respectively) and a chondroblastic osteosarcoma (FIGS. 2G-I). Microscopic examination after H&E staining showed that these three tumor types present similar hypercellular structures and a hyaline cartilage matrix stained pale blue or blue-grey depending on the matrix proteoglycans (FIGS. 2A, B, D, E, G, H). In all three cases, the tumor cells varied in size and shape and contained a hyperchromatic nucleus that was often binucleate. Typical of its higher grade, the conventional chondrosarcoma grade III (FIGS. 2D, E) was more cellular than the conventional chondrosarcoma grade II (FIGS. 2A, B), it contained more atypically sized, shaped and hyperchromatic nuclei, and myxoid changes (a liquefied aspect of the matrix) were also present. In this case of chondroblastic osteosarcoma (FIGS. 2G, H), no osteoid matrix (an essential feature for the diagnosis of osteosarcoma) was observed in the sample. This illustrates the difficulty of distinguishing between chondroblastic osteosarcoma and conventional chondrosarcoma in small biopsy samples: regions of osteoid matrix, essential for the diagnosis of osteosarcoma, may not be present in the tissue sample. In the absence of a marker for osteosarcoma, definitive diagnosis in this case of chondroblastic osteosarcoma can only be performed upon surgical resection of the tumor.

By contrast, immunohistochemistry using anti-GAL1 antibody revealed a clear difference between chondroblastic osteosarcoma and conventional chondrosarcoma: the conventional chondrosarcomas were negative for GAL1 staining whatever the grade (FIGS. 2C, F) whereas chondroblastic osteosarcoma was highly stained (FIG. 2I). GAL1 staining of a papillary thyroid carcinoma served as a positive control. Papillary adenocarcinoma cells have diffuse and strong cell membrane immunoreactivity against GAL1, whereas normal follicular gland cells do not (FIG. 2L). We also analysed dedifferentiated chondrosarcoma to see whether GAL1 immunostaining would differentiate the chondrosarcomatous from the ostesarcomatous components of the tumor. As shown by H&E staining, dedifferentiated chondrosarcoma (FIG. 2J) associates high-grade osteosarcoma (diagnosed by osteoid production forming a thin network between neoplastic cells) with well-differentiated chondrosarcoma. In this case, the chondroid component was not labeled whereas the juxtaposed high-grade osteosarcoma stained intensely for GAL1 (FIG. 2K). All the samples of two other types of chondrosarcoma—mesenchymal chondrosarcoma and clear cell chondrosarcoma—were negative for GAL1 staining (data not shown).

This discriminatory potential of GAL1 staining for osteosarcoma was confirmed by western blotting of representative samples of chondroblastic osteosarcoma and conventional chondrosarcoma from our series. As shown in FIG. 3, the chondroblastic osteosarcomas expressed high levels of GAL1, whereas conventional chondrosarcomas, whatever the grade of malignancy, were negative, confirming the immunohistochemical data. Normal human osteoblasts, human mesenchymal stem cells and the mouse MS5.1 stromal cell line served as positive controls for GAL1 and bone marrow-derived stromal cells from galectin-1 knockout mice served as a negative control (FIG. 3).

Statistical Analysis and Diagnostic Value of GAL1 Expression in Osteosarcoma and Chondrosarcoma

We stained all 165 bone tumors in our collection for GAL1. Table 2 summarizes the percentage of stained cells (divided into six ranges; part A) and the staining intensity (divided into four groups; part B). Two independent pathologists analyzed the GAL1 staining and the concordance of their data was assessed by unweighted Cohen's Kappa coefficient (Ludbrook J, 2002 and Malpica A, et Al., 2005), which measures the agreement between the pathologists and ranges from 1 for perfect concordance to −1 to complete discordance. In our analysis, the Kappa coefficient was 0.75 (p<10⁻⁴) for the percentage of stained cells and 0.88 (p<10⁻⁴) for the staining intensity (data not shown), indicating a high concordance between the pathologists.

We found that GAL1 was expressed in 80 out of 87 cases of osteosarcoma whereas only 15 out of 78 chondrosarcomas expressed GAL1 (Table 2, left columns). GAL1 was expressed in a large proportion of cells and at high intensity in osteosarcomas when compared with chondrosarcomas, the vast majority of which were negative for GAL1 staining. Only four of the chondrosarcomas had more than 50% of cells staines for GAL1 and were dedifferentiated chondrosarcomas. However in theses cases, the GAL1 positive cells were in the high-grade osteosarcomatous component and not in the low-grade chondromatous component.

Similarly, when we compared chondroblastic osteosarcomas with conventional chondrosarcomas, GAL1 was expressed in a relatively large proportion of cells and at high intensity in 22 out of 25 cases of chondroblastic osteosarcoma whereas only 12 out of 66 cases of the conventional chondrosarcoma stained for GAL1 and these were at relatively low intensity (Table 2, right columns). As determined by the Chi² coefficient, there was a significant statistical difference in the percentage of stained cells (Table 2A) and in the intensity of staining (Table 2B) between chondrosarcoma and osteosarcoma (p<10⁻⁴) and between conventional chondrosarcoma and chondroblastic osteosarcoma (p<10⁻³). In addition, the significance of the Cochrane-Armitage trend test (p<10⁻⁴) indicated that the difference in staining between chondrosarcomas and osteosarcomas or between conventional chondrosarcomas and chondroblastic osteosarcomas was robust and would not occur by chance. The Cochrane-Armitage trend test also indicated that the probability that a tumor was a chondroblastic osteosarcoma was higher when both the percentage of stained cells and the staining intensity was high. Indeed, 28% of chondroblastic osteosarcomas fell into to the highest, 76-100% GAL1-positive group compared to less than 2% of conventional chondrosarcomas and, at the other end of the scale, approximately 82% of conventional chondrosarcomas and only 8% of chondroblastic osteosarcomas were not stained by GAL1 (0% staining group). Similarly, when staining intensity was analysed (Table 2B), we found 24% of chondroblastic osteosarcomas stained at the highest intensity (+++ group) compared to 0% of conventional chondrosarcomas and, at the other end of the scale, approximately 82% of conventional chondrosarcomas were completely negative whereas only 8% of chondroblastic osteosarcomas were completely negative.

TABLE 2
Results of GAL1 staining and evaluation of its specificity and sensibility
in osteosarcoma versus chondrosarcoma and chondroblastic chondrosarcoma.

| | | | | | | | Conventional | | Chondroblastic | | | |
|---|---|---|---|---|---|---|---|---|---|---|---|---|
| | Chondrosarcoma | | Osteosarcoma | | | | Condrosarcoma | | osteosarcoma | | | |
| | (CS) | | (OS) | | | | (CC) | | (CO) | | | |
| | N = 78 | | N = 87 | | CS/OS | | N = 66 | | N = 25 | | CC/CO | |
| Groups | Number | % | Number | % | Sensitivity | Specificity | Number | % | Number | % | Sensitivity | Specificity |

A: Number of GAL1 positive cells

| | | | | | | | | | | | | |
|---|---|---|---|---|---|---|---|---|---|---|---|---|
| 0 | 63 | 80.77 | 7 | 8.05 | | | 54 | 81.82 | 2 | 8 | | |
| 1-5 | 4 | 5.13 | 7 | 8.05 | 91.95 | 80.77 | 4 | 6.06 | 2 | 8 | 92 | 81.82 |
| 6-25 | 5 | 6.41 | 11 | 12.64 | 83.91 | 85.9 | 5 | 7.58 | 4 | 16 | 84 | 87.88 |
| 26-50 | 2 | 2.56 | 15 | 17.24 | 71.26 | 92.31 | 2 | 3.03 | 6 | 24 | 68 | 95.45 |
| 51-75 | 2 | 2.56 | 13 | 14.84 | 54.02 | 94.87 | 0 | 0 | 4 | 16 | 44 | 98.48 |
| 76-100 | 2 | 2.56 | 34 | 39.08 | 39.08 | 97.44 | 1 | 1.52 | 7 | 28 | 28 | 98.48 |

| Chi-2 | p < 10−4 | ROC Area = 0.90 [0.86-0.95] | p < 10−3 | ROC Area = 0.92 [0.85-0.98] |
|---|---|---|---|---|
| Trend test | p < 10−4 | | p < 10−4 | |

B: Intensity of GAL1 staining

| | | | | | | | | | | | | |
|---|---|---|---|---|---|---|---|---|---|---|---|---|
| 0 | 63 | 80.77 | 7 | 8.05 | | | 54 | 81.82 | 2 | 8 | | |
| + | 9 | 11.54 | 23 | 26.44 | 91.95 | 80.77 | 9 | 13.64 | 8 | 32 | 92 | 81.32 |
| ++ | 5 | 6.41 | 27 | 31.03 | 65.52 | 92.31 | 3 | 4.55 | 9 | 36 | 60 | 100 |
| +++ | 1 | 1.28 | 30 | 34.48 | 34.48 | 98.72 | 0 | 0 | 6 | 24 | 24 | 100 |

| Chi-2 | p < 10−4 | ROC Area = 0.90 [0.85-0.95] | p < 10−4 | ROC Area = 0.91 [0.84-0.98] |
|---|---|---|---|---|
| Trend test | p < 10−4 | | p < 10−4 | |

The sensitivity is the probability for OS (or CO) to be GAL1 positive (i.e. to have a staining greater than or equal to the considered category). The specificity is the probability for CS (or CC) to be GAL1 negative (i.e. to have a staining lower than the considered category). Chi-square values were used to evaluate the statistical significance of the distribution homogeneity across the groups. The Cochrane Armitage trend tests were used to evaluate trend across the staining ordered groups. P-values are given uncorrected. ROC Area stands for Area under the Receiver Operator Curve and is an overall estimator of the ability of the different categories used as positivity threshold to discriminate OS from CS (or CO from CC).

### REFERENCES

Throughout this application, various references describe the state of the art to which this invention pertains. The disclosures of these references are hereby incorporated by reference into the present disclosure.
Andersen H, Jensen O N, Moiseeva E P, Eriksen E F. A proteome study of secreted prostatic factors affecting osteoblastic activity: galectin-1 is involved in differentiation of human bone marrow stromal cells. J Bone Miner Res. 2003 February; 18(2):195-203.
Barondes S H, Castronovo V, Cooper D N, Cummings R D, Drickamer K, Feizi T, Gitt M A, Hirabayashi J, Hughes C, Kasai K, et al. Galectins: a family of animal beta-galactoside-binding lectins. Cell. 1994 Feb. 25; 76(4):597-8.
Bourne, Y., Bolgiano, B., Liao, D. I., Strecker, G., Cantau, P., Herzberg, O., Feizi, T., and Cambillau, C. 1994. Crosslinking of mammalian lectin (galectin-1) by complex biantennary saccharides. Nat Struct Biol 1:863-870.
Brandt B, Büchse T, Abou-Eladab E F, Tiedge M, Krause E, Jeschke U, Walzel H. Galectin-1 induced activation of the apoptotic death-receptor pathway in human Jurkat T lymphocytes. Histochem Cell Biol. 2008 May; 129(5):599-609. Epub 2008 Feb. 21.
Camby I, Belot N, Lefranc F, Sadeghi N, de Launoit Y, Kaltner H, Musette S, Darro F, Danguy A, Salmon I, Gabius H J, Kiss R. Galectin-1 modulates human glioblastoma cell migration into the brain through modifications to the actin cytoskeleton and levels of expression of small GTPases. J Neuropathol Exp Neurol. 2002 July; 61(7):585-96.
Camby I, Le Mercier M, Lefranc F, Kiss R. Galectin-1: a small protein with major functions. Glycobiology. 2006 November; 16(11):137R-157R. Epub 2006 Jul. 13. Review.
Chiariotti, L, Berlingieri, M T, Battaglia, C, Benvenuto, G, Martelli, M L, Salvatore, P, Chiappetta, G, Bruni, C B, and Fusco, A. 1995. Expression of galectin-1 in normal human thyroid gland and in differentiated and poorly differentiated thyroid tumors. Int J Cancer 64:171-175.
Cindolo, L, Benvenuto, G, Salvatore, P, Pero, R, Salvatore, G, Mirone, V, Prezioso, D, Altieri, V, Bruni, CB, and Chiariotti, L. 1999. galectin-1 and galectin-3 expression in human bladder transitional-cell carcinomas. Int J Cancer 84:39-43.
Drickamer, K. 1988. Two distinct classes of carbohydrate-recognition domains in animal lectins. J Biol Chem 263:9557-9560.
Ellerhorst J, Nguyen T, Cooper D N, Lotan D, Lotan R. Differential expression of endogenous galectin-1 and galectin-3 in human prostate cancer cell lines and effects of overexpressing galectin-1 on cell phenotype. Int J. Oncol. 1999 February; 14(2):217-24.
Elola M T, Wolfenstein-Todel C, Troncoso M F, Vasta G R, Rabinovich G A. Galectins: matricellular glycan-binding proteins linking cell adhesion, migration, and survival. Cell Mol Life Sci. 2007 July; 64(13):1679-700. Review.
Fletcher C D M, U. K., Mertens F. 2002. Pathology and genetics of soft tissue and bone. Lyon: IRC Press.
Gupta, K, Radhika, S, and Vasishta, R. K. 2004. Chondrosarcoma of calcanaeum in a 12-year-old male patient: a case report. Diagn Cytopathol 31:399-401.
Huvos, A G, Rosen, G, and Marcove, R C. 1977. Primary osteogenic sarcoma: pathologic aspects in 20 patients after treatment with chemotherapy en bloc resection, and prosthetic bone replacement. Arch Pathol Lab Med 101:14-18.
Jung E J, Moon H G, Cho B I, Jeong C Y, Joo Y T, Lee Y J, Hong S C, Choi S K, Ha W S, Kim J W, Lee C W, Lee J S, Park S T. Galectin-1 expression in cancer-associated stromal cells correlates tumor invasiveness and tumor progression in breast cancer. Int J. Cancer. 2007 Jun. 1; 120(11):2331-8.
Koh, J S, Chung, J H, Lee, S Y, and Lee, J H. 2001. Chondrosarcoma of the proximal femur with myxoid degeneration mistaken for chondromyxoid fibroma in a young adult. A case report. Acta Cytol 45:254-258.
Koh H S, Lee C, Lee K S, Ham C S, Seong R H, Kim S S, Jeon S H. CD7 expression and galectin-1-induced apoptosis of immature thymocytes are directly regulated by NF-kappaB upon T-cell activation. Biochem Biophys Res Commun. 2008 May 23; 370(1):149-53. Epub 2008 Mar. 18.
Komori, T. 2006. Regulation of osteoblast differentiation by transcription factors. J Cell Biochem 99:1233-1239.
Kopitz J, von Reitzenstein C, André S, Kaltner H, Uhl J, Ehemann V, Cantz M, Gabius H J. Negative regulation of neuroblastoma cell growth by carbohydrate-dependent surface binding of galectin-1 and functional divergence from galectin-3. J Biol Chem. 2001 Sep. 21; 276(38):35917-23. Epub 2001 Jul. 12.
Ludbrook J. Statistical techniques for comparing measurers and methods of measurement: a critical review. Clin Exp Pharmacol Physiol. 2002 July; 29(7):527-36. Review.
Mackie, E J, Ahmed, Y A, Tatarczuch, L, Chen, K S, and Mirams, M. 2008. Endochondral ossification: how cartilage is converted into bone in the developing skeleton. Int J Biochem Cell Biol 40:46-62.
Mahesha, V, Goyal, R, Vaiphei, K, Nada, R, and Gupta, A. K. 2006. Primary chondrosarcoma of ethmoid bone in a 6-year-old child. Ann Diagn Pathol 10:154-156.
Malpica A, Matisic J P, Niekirk D V, Crum C P, Staerkel G A, Yamal J M, Guillaud M H, Cox D D, Atkinson E N, Adler-Storthz K, Poulin N M, Macaulay C A, Follen M. Kappa statistics to measure interrater and intrarater agreement for 1790 cervical biopsy specimens among twelve pathologists: qualitative histopathologic analysis and methodologic issues. Gynecol Oncol. 2005 December; 99(3 Suppl 1):S38-52. Epub 2005 Sep. 23.
Moiseeva E P, Spring E L, Baron J H, de Bono D P. Galectin 1 modulates attachment, spreading and migration of cultured vascular smooth muscle cells via interactions with cellular receptors and components of extracellular matrix. J Vasc Res. 1999 January-February; 36(1):47.
Picci, P. 2007. Osteosarcoma (osteogenic sarcoma). Orphanet J Rare Dis 2:6.
Rorive S, Belot N, Decaestecker C, Lefranc F, Gordower L, Micik S, Maurage C A, Kaltner H, Ruchoux M M, Danguy A, Gabius H J, Salmon I, Kiss R, Camby I. Galectin-1 is highly expressed in human gliomas with relevance for modulation of invasion of tumor astrocytes into the brain parenchyma. Glia. 2001 Mar. 1; 33(3):241-55. Erratum in: Glia 2001 August; 35(2):166.
Rosen, G, Caparros, B, Huvos, A G, Kosloff, C, Nirenberg, A, Cacavio, A, Marcove, R C, Lane, J M, Mehta, B, and Urban, C. 1982. Preoperative chemotherapy for osteogenic sarcoma: selection of postoperative adjuvant chemotherapy based on the response of the primary tumor to preoperative chemotherapy. Cancer 49:1221-1230.
Sanford G L, Harris-Hooker S. Stimulation of vascular cell proliferation by beta-galactoside specific lectins. FASEB J. 1990 August; 4(11):2912-8.
Satelli A, Rao P S, Gupta P K, Lockman P R, Srivenugopal K S, Rao U S. Varied expression and localization of multiple galectins in different cancer cell lines. Oncol Rep. 2008 March; 19(3):587-94.
Sharon, N., and Lis, H. 1989. Lectins as cell recognition molecules. Science 246:227-234.
Thijssen V L, Postel R, Brandwijk R J, Dings R P, Nesmelova I, Satijn S, Verhofstad N, Nakabeppu Y, Baum L G, Bakkers J, Mayo K H, Poirier F, Griffioen A W. Galectin-1 is essential in tumor angiogenesis and is a target for antiangiogenesis therapy. Proc Natl Acad Sci USA. 2006 Oct. 24; 103(43):15975-80. Epub 2006 Oct. 16.
Valenzuela H F, Pace K E, Cabrera P V, White R, Porvari K, Kaija H, Vihko P, Baum L G. O-glycosylation regulates LNCaP prostate cancer cell susceptibility to apoptosis induced by galectin-1. Cancer Res. 2007 Jul. 1; 67(13):6155-62.
Van den Brûle F, Califice S, Castronovo V. Expression of galectins in cancer: a critical review. Glycoconj J. 2004; 19(7-9):537-42. Review.
Xu, X C, el-Naggar, A K, and Lotan, R. 1995. Differential expression of galectin-1 and galectin-3 in thyroid tumors. Potential diagnostic implications. Am J Pathol 147:815-822.

1. A method for diagnosing whether a tumor of a patient is either osteosarcoma or chondrosarcoma comprising the steps of
detecting galectin-1 (GAL1) expression in a bone tumor sample obtained from said patient, and, based on results obtained in said detecting step
determining whether said tumor is an osteosarcoma or a chondrosarcoma.

2-3. (canceled)

4. The method according to claim 1, wherein said GAL1 expression is detected by quantifying a level of GAL1 protein in said bone sample.

5. The method according to claim 4, wherein quantification of the level of GAL1 protein is performed by using a set of antibodies directed against GAL1 protein.

6. The method according to claim 5, wherein said quantification of the level of GAL1 protein is performed by immunohistochemistry.

7. The method according to claim 1, wherein said osteosarcoma is a chondroblastic osteosarcoma and said chondrosarcoma is a conventional chondrosarcoma.

8. The method of claim 6, wherein said step of detecting is carried out by determining an amount of cells in said bone tumor sample that express GAL1.

9. The method of claim 8, wherein osteosarcoma is indicated if at least 20% of cells in said bone tumor sample express GAL1.
